(19) United States
(12) Patent Application Publication
(10) Pub. No.: US 2024/0218463 A1
(43) Pub. Date: Jul. 4, 2024

(54) DNA BARCODE FOR SCREENING FLOCCULARIA LUTEOVIRENS WITH HIGH TOTAL POLYSACCHARIDE CONTENT

(71) Applicant: XIZANG VOCATIONAL TECHNICAL COLLEGE, LHASA (CN)

(72) Inventors: MANJUN YANG, LHASA (CN); CAIXIA ZHANG, LHASA (CN); YISHUI CAI, LHASA (CN)

(21) Appl. No.: 18/418,415

(22) Filed: Jan. 22, 2024

Related U.S. Application Data
(63) Continuation of application No. PCT/CN2022/109999, filed on Aug. 3, 2022.

(30) Foreign Application Priority Data
Nov. 19, 2021 (CN) 202111401727.7

(51) Int. Cl. C12Q 1/6895; C12Q 1/6806; C12Q 1/6858; C12Q 1/686; G01N 27/447

(52) U.S. Cl. CPC C12Q 1/6895; C12Q 1/6806; C12Q 1/6858; C12Q 1/686; G01N 27/44726; C12Q 2600/13

(57) ABSTRACT

The present invention discloses a DNA barcode for screening Floccularia luteovirens with high total polysaccharide content, belonging to the technical field of screening of edible fungi germplasm resources. Compared with a traditional breeding method and other existing DNA barcode technologies, the present invention has the advantages of time saving, labor saving, money saving, accuracy and high efficiency, plays a positive role in genetic breeding of high-quality Floccularia luteovirens, and also provides an effective method for identification and protection of germplasm resources.

### SEQUENCE LISTING

The instant application contains a Sequence Listing which has been submitted electronically in ST.26 format and is hereby incorporated by reference in its entirety. Said ST.26 copy, created on Jan. 18, 2024, is named Sequence Listing.xml and is 17,970 bytes in size.

### TECHNICAL FIELD

The present invention relates to the technical field of screening of edible fungi germplasm resources, in particular to a DNA barcode for screening Floccularia luteovirens with high total polysaccharide content.

### BACKGROUND

Floccularia luteovirens in a gold yellow color, is also known as yellow mushroom and golden mushroom. Floccularia luteovirens is mainly distributed in Qinghai-Tibet Plateau; and the main producing areas include Dangxiong County of Tibet Autonomous Region, Qilian County of Qinghai Province and Shiqu County of Sichuan Province, and the quality of these three main producing areas is the best. Floccularia luteovirens is a high-quality edible fungus with a unique flavor, which cannot be cultivated artificially at present. Main indexes to evaluate the nutritional value, flavor and biological activity of Floccularia luteovirens include, high contents and strong antioxidant activity of total soluble proteins, total soluble amino acids, total polyphenols, total polysaccharides and total fat. It is very difficult to screen high-quality strains by traditional methods. In addition, due to high altitude of main producing areas of the distribution, it is also difficult to collect samples. In order to realize development and utilization of Floccularia luteovirens, it is especially important and urgent to screen high-quality Floccularia luteovirens strains through assistance of a DNA barcode molecular identification technology. In different producing places, Floccularia luteovirens has different nutritional values, different flavors, different biological activities and different market prices. In the past, the breeding of Floccularia luteovirens was mainly determined by a morphological method combined with beneficial content indexes.

However, due to the influence of the special climate environment of Qinghai-Tibet Plateau, the phenomena of different objects with the same name and the same object with different names often appeared to Floccularia luteovirens produced in different areas, so morphological identification was difficult to realize effective distinguishment. More difficultly, it is impossible to screen out high-quality strains with high contents of total soluble proteins, total soluble amino acids, total polyphenols, total polysaccharides and total fat, and strong antioxidant activity by the morphological method. A DNA barcode molecular identification technology is a molecular biology technology based on DNA barcodes (conserved and stable genetic DNA sequences in a genome) to recognize and identify species and excellent quality. It is the effective supplement and expansion of traditional breeding methods, and can accurately and effectively identify samples when samples are incomplete in morphologies or lack morphological structures (processed products such as powder, etc.).

In the existing DNA barcode technology, non-coding regions or conserved gene sequences in ITS (internal transcribed spacer in ribosomal RNA) and mitochondria are mainly used for species object identification; the operation of restriction fragment length polymorphism (RFLP) is very complicated; reliability and repeatability of results are poor; random amplified polymorphic DNA (RAPD) is easily disturbed, which requires a high technical level of operators and is difficult to popularize in assisted breeding; and single nucleotide polymorphism (SNP) has high requirements for equipment and high cost.

Therefore, in view of shortcomings of traditional breeding methods that the breeding of Floccularia luteovirens strains is not accurate enough, time-consuming and labor-consuming, an urgent problem to be solved by those skilled in the art is how to provide a DNA barcode that can accurately and quickly identify a strain of Floccularia luteovirens and realize high-quality breeding, which has the characteristics of low cost, high efficiency, simple operation, stable and reliable results and good repeatability.

### SUMMARY

In view of this, the present invention provides a DNA barcode for screening Floccularia luteovirens with high total polysaccharide content.

In order to achieve the above purpose, the present invention adopts the following technical solution:

A DNA barcode for screening a total polysaccharide content index of Floccularia luteovirens, wherein a nucleotide sequence of the DNA barcode includes one or more of:

a combination of SEQ ID NO:3 and SEQ ID NO:5,
and/or a combination of SEQ ID NO:4 and SEQ ID NO:5,
and/or a combination of SEQ ID NO:3, SEQ ID NO:4 and SEQ ID NO:5,
and/or SEQ ID NO:8,
and/or SEQ ID NO:9,
and/or a combination of SEQ ID NO:8 and SEQ ID NO:9,
and/or SEQ ID NO: 12,
and/or SEQ ID NO: 13,
and/or a combination of SEQ ID NO:12 and SEQ ID NO:13,
and/or a combination of SEQ ID NO:16 and SEQ ID NO:17.

According to the present invention, based on all simple sequence repeats (SSR) in the whole genome of Floccularia luteovirens, fluorescent PCR amplification is carried out; a DNA barcode which effectively corresponds to total polysaccharide content is established, through comparison between the amplified fragments and the DNA barcode of the present invention, quick and accurate screening of Floccularia luteovirens strains with high total polysaccharide content can be realized; and beneficial assistance can be provided for breeding of Floccularia luteovirens.

Another purpose of the present invention is to provide a primer group for amplifying the DNA barcode for screening the total polysaccharide content index of Floccularia luteovirens, wherein a nucleotide sequence of the primer group includes one or more groups of:
SEQ ID NO: 1 and SEQ ID NO:2,
and/or SEQ ID NO:6 and SEQ ID NO:7,
and/or SEQ ID NO:10 and SEQ ID NO: 11,
and/or SEQ ID NO:14 and SEQ ID NO:15.

As a preferred technical solution of the present invention, the nucleotide sequence of the primer group includes: such as SEQ ID NO:1 and SEQ ID NO:2, SEQ ID NO: 6 and SEQ ID NO:7, SEQ ID NO: 10 and SEQ ID NO:11, and SEQ ID NO: 14 and SEQ ID NO:15.

Different primer groups of the present invention can be used alone or in combination to screen the total polysaccharide content of Floccularia luteovirens; and when all primer groups are used together, the screening accuracy is the highest.

Another purpose of the present invention is to provide a method for screening Floccularia luteovirens with a total polysaccharide content index, which comprises the following steps:
S1, extracting genomic DNA of a sample to be tested;
S2, taking the genomic DNA in S1 as a template, and performing a fluorescent PCR amplification reaction on one or more groups of the above primers to obtain an amplification product;
S3, detecting the amplification product of S2 by capillary fluorescence electrophoresis, and judging by the number of fragments, the number of SSR loci, SSR repetitive elements and repeating times of the amplification product.

As a preferred technical solution of the present invention, a judgment standard in step S3 is:
when the primer group of SEQ ID NO:1 and SEQ ID NO:2 is amplified to obtain a 263 bp fragment containing 5 ACC repetitive elements, a 266 bp fragment containing 6 ACC repetitive elements, and a 269 bp fragment containing 7 ACC repetitive elements;
and/or the primer group of SEQ ID NO:6 and SEQ ID NO:7 is amplified to obtain a 255 bp fragment containing 7 TG repetitive elements and a 257 bp fragment containing 8 TG repetitive elements;
and/or the primer group of SEQ ID NO:10 and SEQ ID NO:11 is amplified to obtain a 222 bp fragment containing 6 AT repetitive elements and a 232 bp fragment containing 12 AT repetitive elements;
and/or the primer group of SEQ ID NO: 14 and SEQ ID NO: 15 is amplified to obtain a 217 bp fragment containing 5 GAT repetitive elements and a 223 bp fragment containing 7 GAT repetitive elements, the Floccularia luteovirens is judged as Floccularia luteovirens with high total polysaccharide content.

As a preferred technical solution of the present invention, a reaction system of the fluorescent PCR amplification reaction in step S2 is.

5 μL of 2×Taq PCR Master Mix, 1 μL of genomic DNA, 0.1 μL of forward primer, 0.4 μL of reverse primer, and 0.4 μL of M13 primer with fluorescence, wherein a volume is fixed to 10 μL with sterile deionized water.

Further preferably, the concentrations of the forward primer, the reverse primer and the M13 primer with fluorescence are all 10 uM.

As a preferred technical solution of the present invention, a fluorescent PCR amplification reaction procedure in step S2 is:
performing pre-denaturation at 95° C. for 3 min, denaturation at 95° ° C. for 30 s, PCR annealing during the decrease from 62° ° C. to 55° C. for 30 s, and extension at 72° C. for 30 s, with a total of 10 cycles; performing denaturation at 95° C. for 30 s, annealing at 52° C. for 30 s, and extension at 72° C. for 30 s, with a total of 25 cycles; performing final extension at 72° C. for 20 min; and after heat preservation at 4° C. for 6 h, using the product for fluorescence capillary electrophoresis detection.

Further another purpose of the present invention is to provide application of the DNA barcode and/or the primer group in preparation of a product for screening Floccularia luteovirens with a total polysaccharide content index.

Further another purpose of the present invention is to provide a product for screening Floccularia luteovirens with a total polysaccharide content index, which comprises one or more of the above primer groups and satisfies one or more of the following standards:
the primer group of SEQ ID NO:1 and SEQ ID NO:2 is amplified to obtain a 263 bp fragment containing 5 ACC repetitive elements, a 266 bp fragment containing 6 ACC repetitive elements, and a 269 bp fragment containing 7 ACC repetitive elements;
and/or the primer group of SEQ ID NO:6 and SEQ ID NO:7 is amplified to obtain a 255 bp fragment containing 7 TG repetitive elements and a 257 bp fragment containing 8 TG repetitive elements;
and/or the primer group of SEQ ID NO: 10 and SEQ ID NO: 11 is amplified to obtain a 222 bp fragment containing 6 AT repetitive elements and a 232 bp fragment containing 12 AT repetitive elements;
and/or the primer group of SEQ ID NO:14 and SEQ ID NO:15 is amplified to obtain a 217 bp fragment containing 5 GAT repetitive elements and a 223 bp fragment containing 7 GAT repetitive elements.

As a preferred technical solution of the present invention, the product is a kit.

According to the above technical solution, compared with the prior art, the present invention discloses and provides the DNA barcode for screening Floccularia luteovirens with high total polysaccharide content. Compared with the prior art, the present invention can accurately and quickly identify Floccularia luteovirens strains and realize a DNA barcode technology of high-quality breeding. The present invention is characterized by low cost, high efficiency, simple operation, stable and reliable results and good repeatability.

Compared with a traditional breeding method and other existing DNA barcode technologies, the present invention has the advantages of time saving, labor saving, money saving, accuracy and high efficiency, plays a positive role in original place identification and genetic breeding of high-quality Floccularia luteovirens, and also provides an effective method for identification and protection of germplasm resources.

### DESCRIPTION OF DRAWINGS

To more clearly describe the technical solutions in the embodiments of the present invention or in the prior art, the drawings required to be used in the description of the embodiments or the prior art will be simply presented below. Apparently, the drawings in the following description are merely the embodiments of the present invention, and for those ordinary skilled in the art, other drawings can also be obtained according to the provided drawings without contributing creative labor.

FIG. 1 is a comparison result diagram of total polysaccharide content in embodiment, reference example 1 and reference example 2 according to the present invention, wherein reference example 1, reference example 2 and embodiment are listed in order from left to right;

FIG. 2A is a diagram of PCR amplification using primer 1 in reference example 1 according to the present invention;

FIG. 2B is a diagram of PCR amplification using primer 1 in reference example 2 according to the present invention;

FIG. 2C is a diagram of PCR amplification using primer 1 in one embodiment according to the present invention;

FIG. 3A is a diagram of PCR amplification using primer 2 in reference example 1 according to the present invention;

FIG. 3B is a diagram of PCR amplification using primer 2 in reference example 2 according to the present invention;

FIG. 3C is a diagram of PCR amplification using primer 2 in one embodiment according to the present invention;

FIG. 4A is a diagram of PCR amplification using primer 3 in reference example 1 according to the present invention;

FIG. 4B is a comparison result diagram of PCR amplification using primer 3 in reference example 2 according to the present invention;

FIG. 4C is a diagram of PCR amplification using primer 3 in one embodiment according to the present invention;

FIG. 5A is a diagram of PCR amplification using primer 4 in reference example 1 according to the present invention;

FIG. 5B is a diagram of PCR amplification using primer 4 in reference example 2 according to the present invention; and

FIG. 5C is a diagram of PCR amplification using primer 4 in one embodiment according to the present invention.

### DETAILED DESCRIPTION

The technical solutions in the embodiments of the present invention will be clearly and fully described below in combination with the drawings in the embodiments of the present invention. Apparently, the described embodiments are merely part of the embodiments of the present invention, not all of the embodiments. Based on the embodiments in the present invention, all other embodiments obtained by those ordinary skilled in the art without contributing creative labor will belong to the protection scope of the present invention.

Embodiments of the present invention disclose a DNA barcode for screening Floccularia luteovirens with high total polysaccharide content.

### Embodiment 1

Establishment of a DNA Barcode of Floccularia luteovirens

Samples of Floccularia luteovirens are collected from Dangxiong County of Tibet Autonomous Region, Qilian County of Qinghai Province and Shiqu County of Sichuan Province and are treated by genome sequencing; and SSR loci in the genome sequences are analyzed by MISA program.

Primers are designed for PCR amplification of the SSR loci; the primers that can amplify the corresponding fragments are reserved; and invalid primers are discarded.

Samples of Floccularia luteovirens from Dangxiong County of Tibet Autonomous Region, Qilian County of Qinghai Province and Shiqu County of Sichuan Province are selected for testing of total polysaccharide content.

The samples from the above three original places are amplified by effective primers and detected by capillary electrophoresis. The simple sequence repeat (SSR) locus corresponding to a total polysaccharide content is established by analysis. Finally, four pairs of primers (see Table 1) are obtained. The four pairs of primers are used to amplify the sample genome; and the fragment polymorphism obtained can assist in screening Floccularia luteovirens with a high total polysaccharide content.

TABLE 1
Specific primers for screening of Floccularia luteovirens strains with high
total polysaccharide content

| | | Denaturation | | Denaturation | |
|---|---|---|---|---|---|
| | | temperature | | temperature | SSR |
| No. | Forward primer | (° C.) | Reverse primer | (° C.) | element |
| 1 | TGTAAAACGACGGC | 60,179 | GGTGATTCGT | 59.965 | ACC |
| | CAGTCACCGCACCAT | | CAGTGTGGG | | |
| | CTGCTGATA, as shown | | T, as shown in | | |
| | in SEQ ID No: 1 | | SEQ ID No: 2 | | |
| 2 | TGTAAAACGACGGC | 59.895 | GAAAGGAAG | 60.11 | TG |
| | CAGTTAACTATTTCC | | GTTCGCGCTT | | |
| | CGGCTCGGC, as | | G, as shown in | | |
| | shown in SEQ ID No: 6 | | SEQ ID No: 7 | | |
| 3 | TGTAAAACGACGGC | 60.108 | AGACGTCAA | 60.109 | TA |
| | CAGTAGCATTCCAGA | | GATGGACTG | | |
| | GACGTCAGC, as | | CG, as shown in | | |
| | shown in SEQ ID No: 10 | | SEQ ID No: 11 | | |
| 4 | TGTAAAACGACGGC | 60.036 | TGGAAGGGA | 60.033 | GAT |
| | CAGTGAAGTGGTAG | | TAGCGAGGT | | |
| | TGGGACTGGC, as | | GA, as shown in | | |
| | shown in SEQ ID No: 14 | | SEQ ID No: 15 | | |

### Embodiment 2

SSR Specific Primer Amplification of Floccularia luteovirens Strains with High Total Polysaccharide Content

### (1) Extraction and Identification of Total Polysaccharides

Samples from Shiqu County of Sichuan Province are taken as test example of the present invention. Freeze-dried powder of fruiting body samples is sieved by a 50-mesh sieve; 20 mL of double distilled water is added into 1 g of dry powder; extraction lasts for 30 min with assistance of 300 W ultrasonic waves; centrifugation of 30 min is carried out at 5000 r/min; and the supernate is taken and prepared into a total polysaccharide extracting solution.

Reference example 1: samples from Dangxiong County of Tibet Autonomous Region (treatment method is the same as above).

Reference example 2: samples from Qilian County of Qinghai Province (treatment method is the same as above).

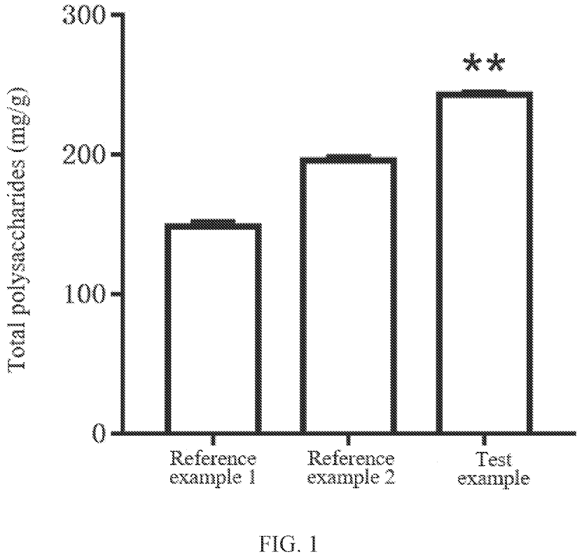

Total polysaccharide content in the extract is determined by a phenol-sulfuric acid method. Please refer to Zhao Qiduo et al. (Journal of Yichun University, 2011, 33(08):74-76) for details. The result is converted to mg/g. The total polysaccharide content in Floccularia luteovirens from Shiqu County of Sichuan Province is 242.88(±2.43) mg/g, which is determined as a test example; the total polysaccharide content in Floccularia luteovirens from Dangxiong County of Tibet Autonomous Region is 148.49(±3.85) mg/g, which is determined as reference example 1; and the total polysaccharide content in Floccularia luteovirens from Qilian County of Qinghai Province is 195.95(±3.28) mg/g, which is determined as reference example 2 (see FIG. 1).
(2) The genome of Floccularia luteovirens samples is extracted by an Ezup column fungus genomic DNA extraction kit (No. B518259) of Sangon Biotech (Shanghai) Co., Ltd, and diluted to 20 ng/μL for fluorescent PCR amplification.
(3) Fluorescent PCR amplification of the SSR DNA barcode is carried out with primers in Table 1.

Fluorescent PCR amplification reaction system (10 μL): 5 μL of 2×Taq PCR MasterMix, 1 μL of template (genomic DNA), 0.1 μL of forward primer, 0.4 μL of reverse primer (concentrations of the forward primer and the reverse primer are both 10 uM), and 0.4 μL of M13 primer with fluorescence (concentration of 10 uM), wherein a volume is fixed to 10 μL with sterile deionized water.

Reaction conditions: performing pre-denaturation at 95° C. for 3 min, denaturation at 95° C. for 30 s, PCR annealing during the decrease from 62° C. to 55° C. for 30 s, and extension at 72° C. for 30 s, with a total of 10 cycles; performing denaturation at 95° ° C. for 30 s, annealing at 52° C. for 30 s, and extension at 72° C. for 30 s, with a total of 25 cycles; performing final extension at 72° C. for 20 min; and after heat preservation at 4° C. for 6 h, using the product for fluorescence capillary electrophoresis detection.
(4) After quantitative dilution of the PCR product, 1 μL of a PCR diluted products is denatured with 9 μL of formamide (including 1% internal standard), and then subjected to capillary fluorescence electrophoresis detection by a DNA sequencer ABI 3730×1. The internal standard, which is a molecular weight internal standard (also known as internal lane standard) LIZ-500 bp, is composed of 16 double-stranded DNA fragments labeled with LIZ fluorescein (orange), with molecular weights of 35, 50, 75, 100, 139, 150, 160, 200, 250, 300, 340, 350, 400, 450, 490 and 500 bp respectively. The fragment size in the amplification result electropherogram is equal to the actual bp number of the amplified fragment plus the M13 fluorescent primer about 18 bp (with error of 1-2 bp). The amplified capillary electrophoresis peak is combined with the sequencing result; and the peak number indicates the number of heterozygous amplified fragments of the gene.
(5) The above methods are used to identify Floccularia luteovirens in test example 1, reference example 1 and reference example 2.

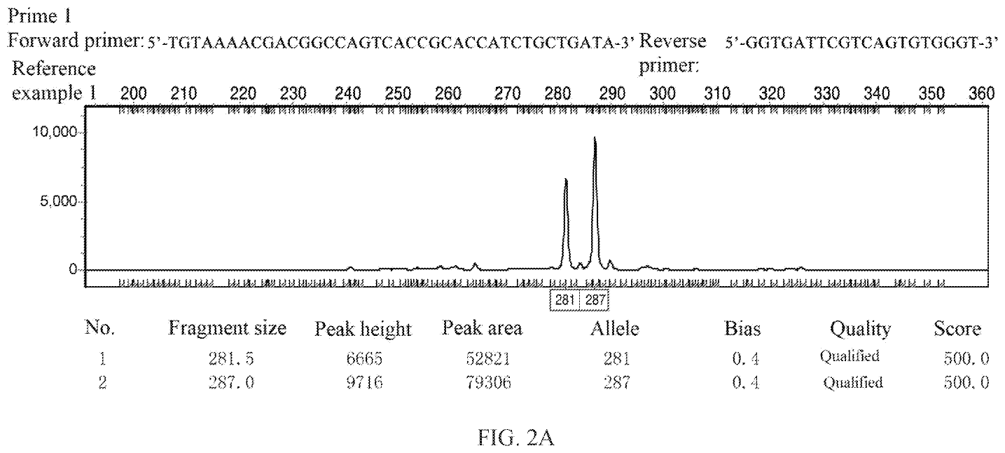

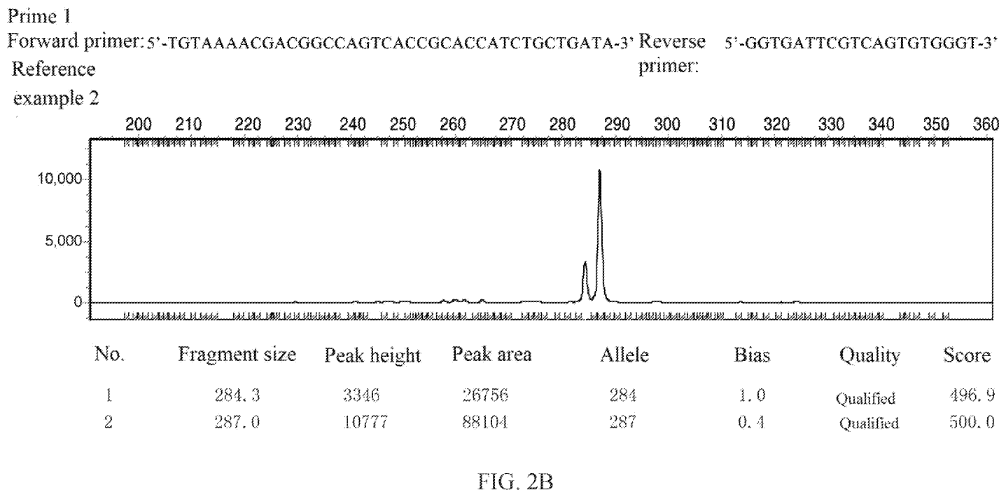

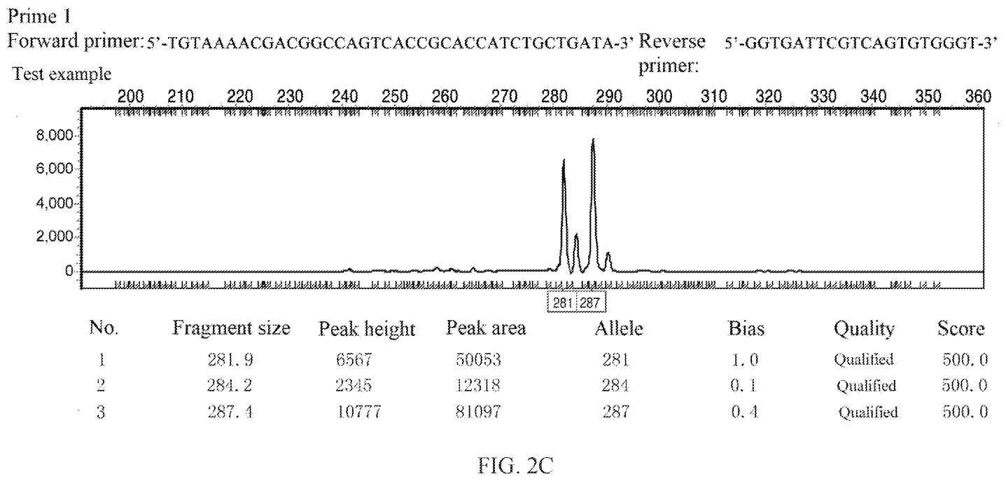

Amplification results of primer 1 are shown in FIGS. 2A-2C. When primer 1 is used for fluorescent PCR amplification, three fragments (three peaks) are obtained through amplification, which contain three SSR loci; and the SSR repetitive element is ACC. The amplified fragment obtained in the test example is characterized by containing a 263 bp fragment with 5 repeats and a 269 bp fragment with 7 repeats.

Amplified fragments of primer 1: (the statistical fragment length of electropherogram includes the M13 fluorescent primer; the specific sequence shows that the M13 fluorescent primer sequence (18 bp) is removed; and the underlined part is an SSR repetitive element.)

263 bp amplified fragment sequence:

(as shown in SEQ ID NO: 3)
CACCGCACCATCTGCTGATAATGAATACTTGTACCACCACCACCACCAA
CACCACGCCCACTCGTTTTCCTGCGTTTCCCTGCGGCACATTTTCCTCA
ATCACAATCACAGTCACCGCTTCCACAGTCCCTCCTAGTCTTGATGTTG
CTCATTTCCCTGTGTGGTCTCGTTTCTTGGTACACACAGGACCGCCACC
GACCTCCCGCCACCTCTCCGTCATTTAACTTCCCGTTATATGCTCATAC
CCACACTGACGAATCACC;

266 bp amplified fragment sequence:

(as shown in SEQ ID NO: 4)
CACCGCACCATCTGCTGATAATGAATACTTGTACCACCACCACCACCAC
CAACACCACGCCCACTCGTTTTCCTGCGTTTCCCTGCGGCACATTTTCC
TCAATCACAATCACAGTCACCGCTTCCACAGTCCCTCCTAGTCTTGATG
TTGCTCATTTCCCTGTGTGGTCTCGTTTCTTGGTACACACAGGACCGCC
ACCGACCTCCCGCCACCTCTCCGTCATTTAACTTCCCGTTATATGCTCA
TACCCACACTGACGAATCACC;

269 bp amplified fragment sequence:

(as shown in SEQ ID NO: 5)
CACCGCACCATCTGCTGATAATGAATACTTGTACCACCACCACCACCAC
CACCAACACCACGCCCACTCGTTTTCCTGCGTTTCCCTGCGGCACATTT
TCCTCAATCACAATCACAGTCACCGCTTCCACAGTCCCTCCTAGTCTTG
ATGTTGCTCATTTCCCTGTGTGGTCTCGTTTCTTGGTACACACAGGACC
GCCACCGACCTCCCGCCACCTCTCCGTCATTTAACTTCCCGTTATATGC
TCATACCCACACTGACGAATCACC

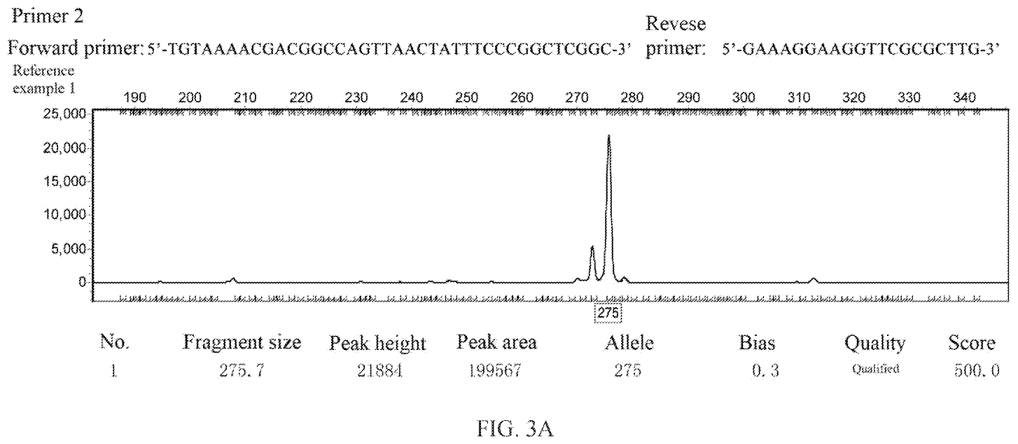

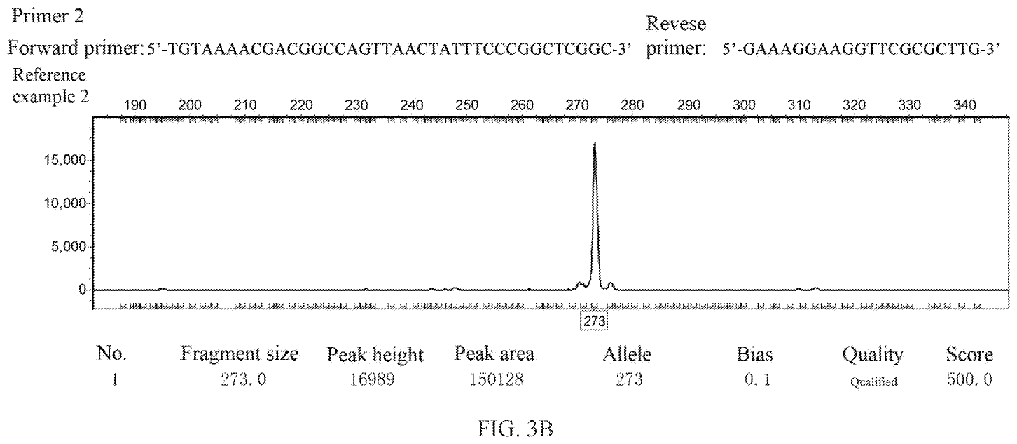

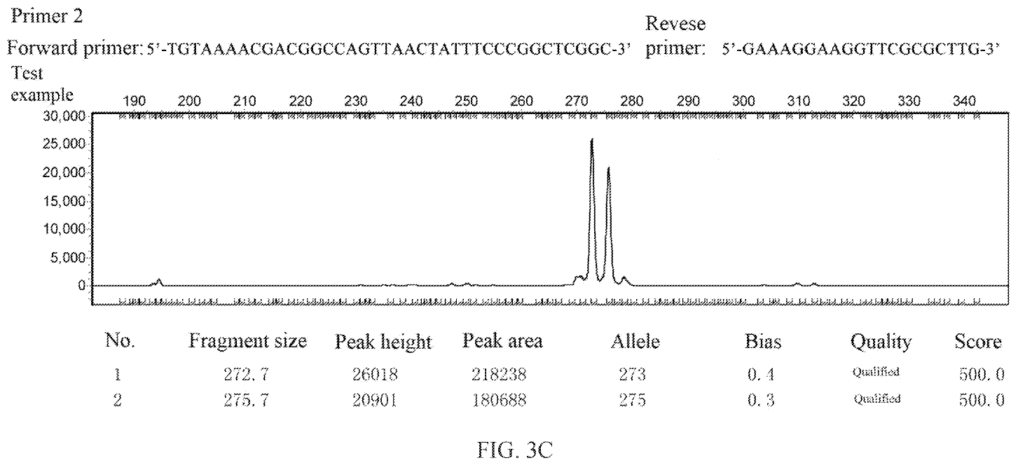

Amplification results of primer 2 are shown in FIGS. 3A-3C. When primer 2 is used for fluorescent PCR amplification, two fragments (two peaks) are obtained, which contain two SSR loci; and the SSR repetitive element is TG. The amplified fragment obtained in the test example is characterized by containing a 255 bp fragment with 7 repeats of TG and a 257 bp fragment with 8 repeats of TG.

Amplified fragments of primer 2: (the statistical fragment length of electropherogram includes the M13 fluorescent primer; the specific sequence shows that the M13 fluorescent primer sequence (18 bp) is removed; and the underlined part is an SSR repetitive element.)

255 bp amplified fragment sequence:

(as shown in SEQ ID NO: 8)
TAACTATTTCCCGGCTCGGCTCTGCAGACAAATTTCAGGGTGTTCCTTT
TCCTGATCCATACATGCCGAACCACACCTGTGACCTGCCGTGCACTATG
CCGATAAACTTCATCGTTTATCCAAGTCTCTCCCACAGGAGTTATATTC
GAGACTCCGTGTAAACCTTATGTACGTCACAAGTATGCCAAGTAGTCAG
CTTCTTCGCATGTGTGTGTGTGTGCGGTTTGTGCTAAATCAAGCGCGAA
CCTTCCTTTC;

257 bp amplified fragment sequence:

(as shown in SEQ ID NO: 9)
TAACTATTTCCCGGCTCGGCTCTGCAGACAAATTTCAGGGTGTTCCTTT
TCCTGATCCATACATGCCGAACCACACCTGTGACCTGCCGTGCACTATG
CCGATAAACTTCATCGTTTATCCAAGTCTCTCCCACAGGAGTTATATTC
GAGACTCCGTGTAAACCTTATGTACGTCACAAGTATGCCAAGTAGTCAG
CTTCTTCGCATGTGTGTGTGTGTGTGCGGTTTGTGCTAAATCAAGCGCG
AACCTTCCTTTC.

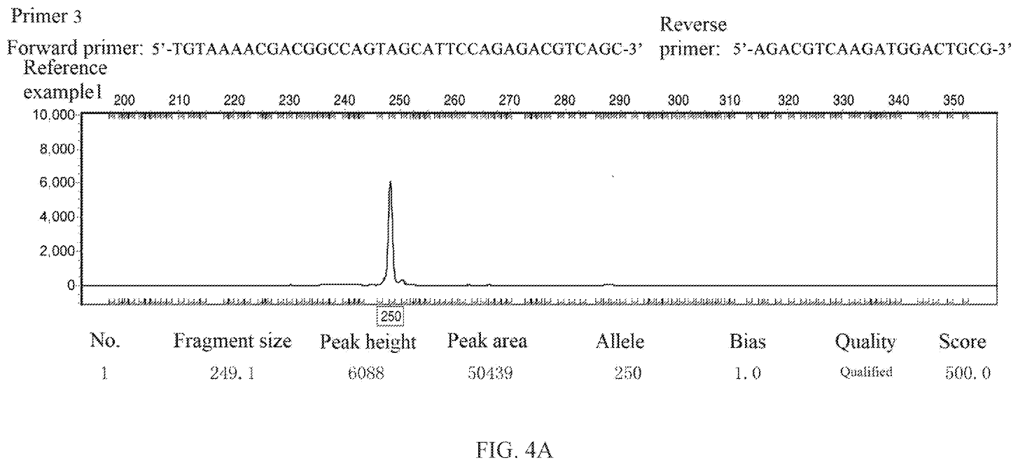

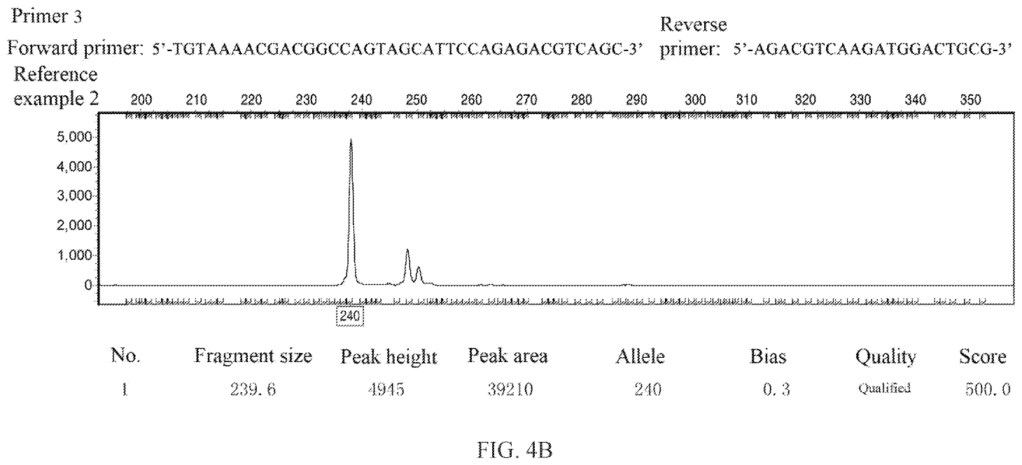

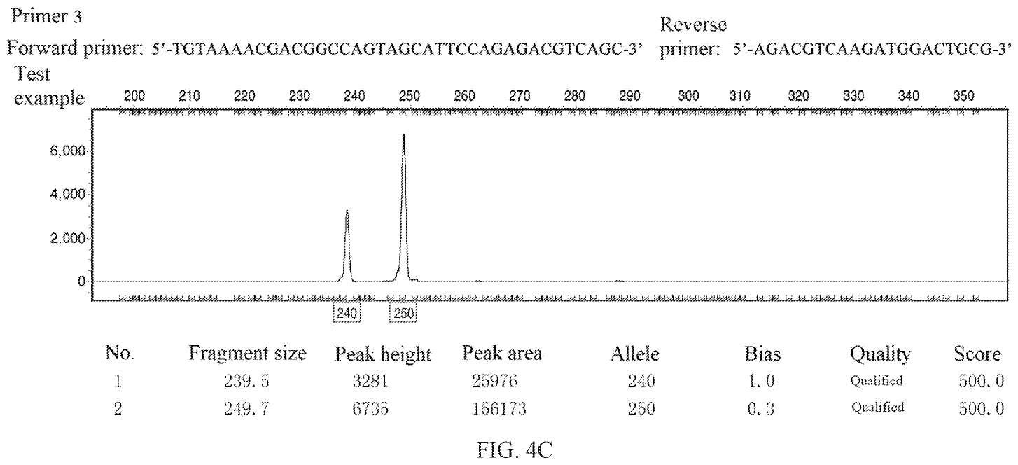

Amplification results of primer 3 are shown in FIGS. 4A-4C. When primer 3 is used for fluorescent PCR amplification, two fragments (two peaks) are obtained, which contain two SSR loci; and the SSR repetitive element is TA. The amplified fragment obtained in the test example is characterized by containing a 222 bp fragment with 6 repeats of AT and a 232 bp fragment with 12 repeats of AT.

Amplified fragments of primer 3: (the statistical fragment length of electropherogram includes the M13 fluorescent primer; the specific sequence shows that the M13 fluorescent primer sequence (18 bp) is removed; and the underlined part is an SSR repetitive element.) 222 bp amplified fragment sequence:

(as shown in SEQ ID NO: 12)
AGCATTCCAGAGACGTCAGCGTCTGTCATATTGATAAAGTTAAAGTAAT
TTGTGCTTGAAGTCATATCATATAACTATTCGGCAGCTCAAAGACTATG
AATGTGCTTACCGGTACCCTAGAGTATATTGAATAAGATTGAATTATAT
ATATATACATATATAATAATGGAACGCACACATATCCGTATTCAGAGCG
ATGATACGCAGTCCATCTTGACGTCT;

232 bp amplified fragment sequence:

(as shown in SEQ ID NO: 13)
AGCATTCCAGAGACGTCAGCGTCTGTCATATTGATAAAGTTAAAGTAAT
TTGTGCTTGAAGTCATATCATATAACTATTCGCAGCTCAAAGACTATGA
ATGTGCTTACCGGTACCCTAGAGTATATTGAATAAGATTGAATTATATA
TATATATATATATATATACATATATAATAATGAACGCACACATATCCGT
ATTCAGAGCGATGATACGCAGTCCATCTTGACGTCT.

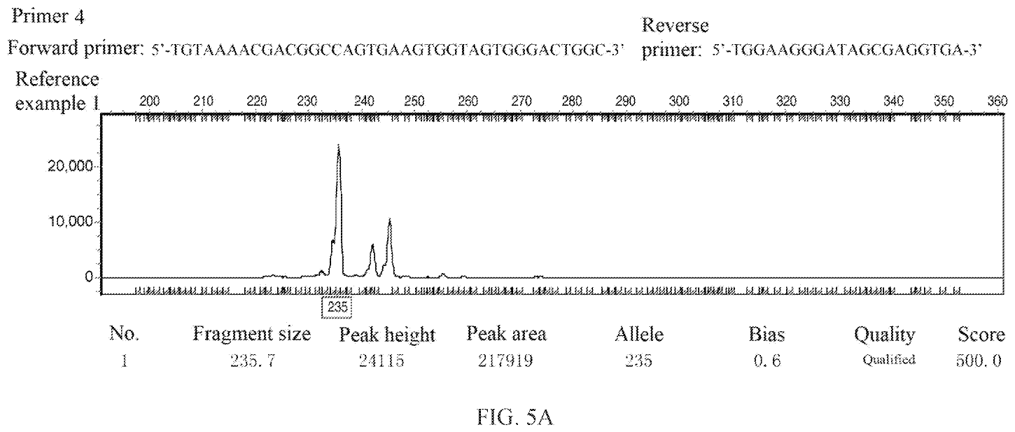

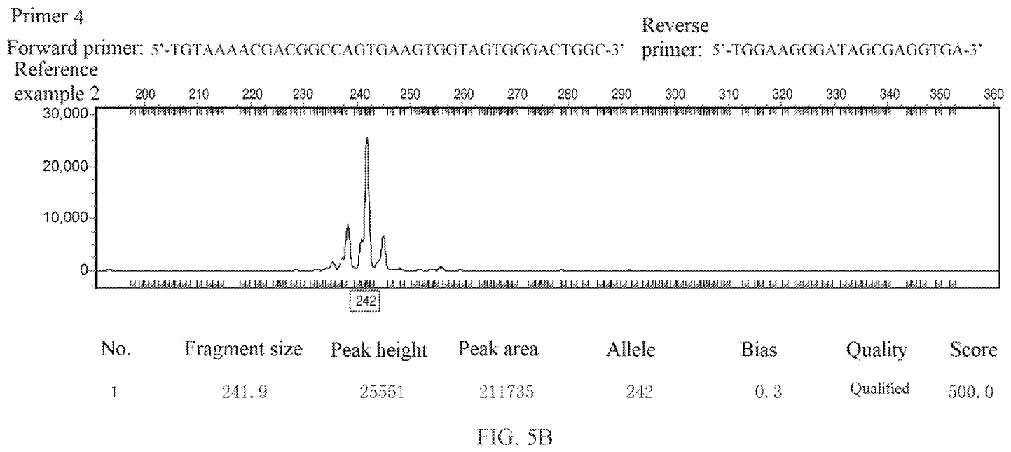

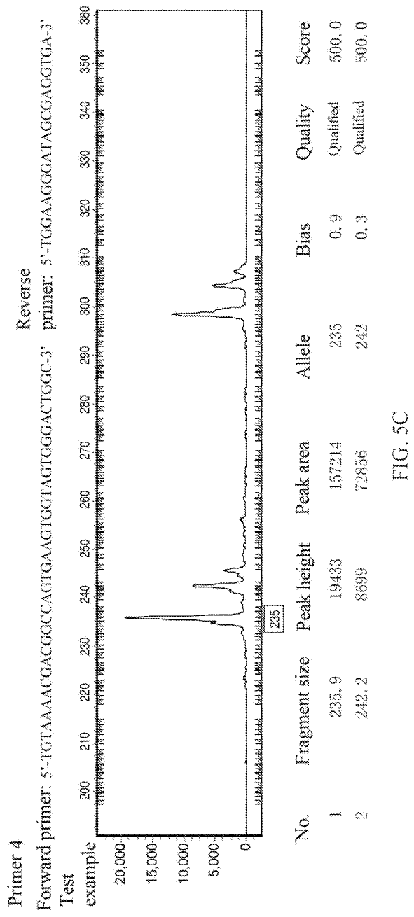

Amplification results of primer 4 are shown in FIGS. 5A-5C. When primer 4 is used for fluorescent PCR amplification, two fragments (two peaks) are obtained, which contain two SSR loci; and the SSR repetitive element is GAT. The amplified fragment obtained in the test example is characterized by containing a 217 bp fragment with 5 repeats and a 223 bp fragment with 7 repeats.

Amplified fragments of primer 4: (the statistical fragment length of electropherogram includes the M13 fluorescent primer; the specific sequence shows that the M13 fluorescent primer sequence (18 bp) is removed; the electropherogram statistical fragment of the 223 bp fragment contains the M13 fluorescent primer; the specific sequence shows that the M13 fluorescent primer sequence (19 bp) with error of 1 bp is removed; and the underlined part is an SSR repetitive element.)

217 bp amplified fragment sequence:

(as shown in SEQ ID NO: 16)
GAAGTGGTAGTGGGACTGGCGAAGAAGTCAGCGAAACCCGGAGGAAGGA
GGAAGAAGAGGAAGGAGGAGGATGTAGGAGATGATGATGAAGAGGAAGA
TGATGATGATGATGACGATGATGATGAACTTTGGAGAGGAAGAACATCC
ATCTTTCCGGACGCGCTTCTGGCTCGATGTCCAGTCTCGGCCACCCAAT
TTCACCTCGCTATCCCTTCCA;

223 bp amplified fragment sequence:

(as shown in SEQ ID NO: 17)
GAAGTGGTAGTGGGACTGGCGAAGAAGTCAGCGAAACCCGGAGGAAGGA
GGAAGAAGAGGAAGGAGGAGGATGTAGGAGATGATGATGAAGAGGAAGA
TGATGATGATGATGATGATGACGATGATGATGAACTTTGGAGAGGAAGA
ACATCCATCTTTCCGGACGCGCTTCTGGCTCGATGTCCAGTCTCGGCCA
CCCAATTTCACCTCGCTATCCCTTCCA.

Through comprehensive analysis of maps and sequencing results of test example, reference example 1 and reference example 2, the characteristic information of the DNA barcode of Floccularia luteovirens with high total polysaccharide content is shown in Table 2.

TABLE 2
DNA barcode characteristics of Floccularia luteovirens with high total
polysaccharide content

| | Primer 1 | Primer 2 | Primer 3 | Primer 4 |
|---|---|---|---|---|
| Total | amplified | amplified | amplified | amplified |
| polysaccharide | fragment | fragment | fragment | fragment |
| content | (SSR) | (SSR) | (SSR) | (SSR) |
| Reference | 263 bp (5ACC) | 257 bp (8TG) | 232 bp (12AT) | 217 bp (5GAT) |
| example 1 | 269 bp (7ACC) | | | |
| Low content | | | | |
| Reference | 266 bp (6ACC) | 255 bp (7TG) | 222 bp (6AT) | 223 bp (7GAT) |
| example 2 | 269 bp (7ACC) | | | |
| Low content | | | | |
| Test example | 263 bp (5ACC) | 255 bp (7TG) | 222 bp (6AT) | 217 bp (5GAT) |
| High content | 266 bp (6ACC) | 257 bp (8TG) | 232 bp (12AT) | 223 bp (7GAT) |
| | 269 bp (7ACC) | | | |

Primer 1 carries out amplification to obtain the 263 bp fragment containing 5 ACC repetitive elements (as shown in SEQ ID NO:3), the 266 bp fragment containing 6 ACC repetitive elements (as shown in SEQ ID NO:4) and the 269 bp fragment containing 7 ACC repetitive elements (as shown in SEQ ID NO:5); primer 2 carries out amplification to obtain the 255 bp fragment containing 7 TG repetitive elements (as shown in SEQ ID NO:8) and the 257 bp fragment containing 8 TG repetitive elements (as shown in SEQ ID NO:9); primer 3 carries out amplification to obtain the 222 bp fragment containing 6 AT repetitive elements (as shown in SEQ ID NO:12) and the 232 bp fragment containing 12 AT repetitive elements (as shown in SEQ ID NO:13); and primer 4 carries out amplification to obtain the 217 bp fragment containing 5 GAT repetitive elements (as shown in SEQ ID NO:16) and the 223 bp fragment containing 7 GAT repetitive elements (as shown in SEQ ID NO:17).

In addition, when primers 1, 2, 3 and 4 are used for comprehensive detection and judgment respectively, the accuracy of screening the total polysaccharide content index of Floccularia luteovirens is best.

### Embodiment 3

Verification of Screening of Total Polysaccharide Content Index of Floccularia luteovirens

A DNA barcode of a total polysaccharide content of Floccularia luteovirens is verified by blind testing.
step 1, blind testing: taking samples from Shiqu County of Sichuan Province with the total polysaccharide content higher than or equal to 242.88 mg/g as test group; taking samples from Dangxiong County of Tibet Autonomous Region and Qilian County of Qinghai Province with the total polysaccharide content lower than 242.88 mg/g (significance p<0.05) as reference group 1 and reference group 2; and taking 16 samples respectively for blind testing;
step 2, testing: using primers (SEQ ID NO:1 and SEQ ID NO:2, SEQ ID NO:6 and SEQ ID NO:7, SEQ ID NO:10 and SEQ ID NO:11, and SEQ ID NO:14 and SEQ ID NO:15) for amplification and capillary electrophoresis. One or more pairs of primer groups can be combined for amplification; and DNA barcode characteristics can be used to distinguish the blind testing samples;
step 3, unblinding: results are shown in Table 3. The unblinding results of 48 samples for distinguishing total polysaccharide content with the total polysaccharide content barcode characteristics are all correct, indicating that the DNA barcode of the total polysaccharide content is suitable for screening the total polysaccharide content character.

TABLE 3
Unblinding identification results based on DNA barcode characteristics of total polysaccharide content

| | Primer 1 | Primer 2 | Primer 3 | Primer 4 | | |
|---|---|---|---|---|---|---|
| Blind | amplified | amplified | amplified | amplified | Unblinding | Identification |
| testing No. | fragment (SSR) | fragment (SSR) | fragment (SSR) | fragment (SSR) | No. | results |
| 2, 4, 5, 7, 8, 12, | 263 bp (5ACC) | 257 bp (8TG) | 232 bp (12AT) | 217 bp (5GAT) | 33-48 | Low total polysaccharide |
| 14, 15, 19, 22, 25, | 269 bp (7ACC) | | | | | content in reference |
| 26, 34, 35, 43, 45 | | | | | | group 1 |
| 3, 10, 11, 18, 21, 23, | 266 bp (6ACC) | 255 bp (7TG) | 222 bp (6AT) | 223 bp (7GAT) | 1-16 | Low total polysaccharide |
| 27, 29, 31, 32, 36, | 269 bp (7ACC) | | | | | content in reference |
| 37, 40, 41, 44, 48 | | | | | | group 2 |
| 1, 6, 9, 13, 16, 17, | 263 bp (5ACC) | 255 bp (7TG) | 222 bp (6AT) | 217 bp (5GAT) | 17-32 | High total polysaccharide |
| 20, 24, 28, 30, 33, | 266 bp (6ACC) | 257 bp (8TG) | 232 bp (12AT) | 223 bp (7GAT) | | content in test group |
| 38, 39, 42, 46, 47 | 269 bp (7ACC) | | | | | |

Each embodiment in the description is described in a progressive way. The difference of each embodiment from each other is the focus of explanation. The same and similar parts among all of the embodiments can be referred to each other.

The above description of the disclosed embodiments enables those skilled in the art to realize or use the present invention. Many modifications to these embodiments will be apparent to those skilled in the art. The general principle defined herein can be realized in other embodiments without departing from the spirit or scope of the present invention. Therefore, the present invention will not be limited to these embodiments shown herein, but will conform to the widest scope consistent with the principle and novel features disclosed herein.

What is claimed is:

1. A DNA barcode for screening a total polysaccharide content index of Floccularia luteovirens, wherein a nucleotide sequence of the DNA barcode comprises one or more of:
a combination of SEQ ID NO:3 and SEQ ID NO:5,
and/or a combination of SEQ ID NO:4 and SEQ ID NO:5,
and/or a combination of SEQ ID NO:3, SEQ ID NO:4 and SEQ ID NO:5,
and/or SEQ ID NO:8,
and/or SEQ ID NO:9,
and/or a combination of SEQ ID NO:8 and SEQ ID NO:9,
and/or SEQ ID NO:12,
and/or SEQ ID NO:13,
and/or a combination of SEQ ID NO:12 and SEQ ID NO:13,
and/or a combination of SEQ ID NO:16 and SEQ ID NO:17.

2. A primer group for amplifying the DNA barcode for screening the total polysaccharide content index of Floccularia luteovirens according to claim 1, wherein a nucleotide sequence of the primer group comprises one or more groups of:
SEQ ID NO: 1 and SEQ ID NO:2,
and/or SEQ ID NO:6 and SEQ ID NO:7,
and/or SEQ ID NO:10 and SEQ ID NO:11,
and/or SEQ ID NO:14 and SEQ ID NO:15.

3. The primer group according to claim 2, wherein the nucleotide sequence of the primer group comprises: such as SEQ ID NO:1 and SEQ ID NO:2, SEQ ID NO: 6 and SEQ ID NO:7, SEQ ID NO: 10 and SEQ ID NO:11, and SEQ ID NO:14 and SEQ ID NO:15.

4. A method for screening Floccularia luteovirens with a total polysaccharide content index, comprising the following steps:
S1, extracting genomic DNA of a sample to be tested;
S2, taking the genomic DNA in S1 as a template, and performing a fluorescent PCR amplification reaction on one or more of the primer groups according to claim 2 to obtain an amplification product;
S3, detecting the amplification product of S2 by capillary fluorescence electrophoresis, and judging by the number of fragments, the number of SSR loci, SSR repetitive elements and repeating times of the amplification product.

5. The method for screening Floccularia luteovirens with the total polysaccharide content index according to claim 4, wherein a judgment standard in step S3 is:
when the primer group of SEQ ID NO:1 and SEQ ID NO:2 is amplified to obtain a 263 bp fragment containing 5 ACC repetitive elements, a 266 bp fragment containing 6 ACC repetitive elements, and a 269 bp fragment containing 7 ACC repetitive elements;
and/or the primer group of SEQ ID NO:6 and SEQ ID NO:7 is amplified to obtain a 255 bp fragment containing 7 TG repetitive elements and a 257 bp fragment containing 8 TG repetitive elements;
and/or the primer group of SEQ ID NO:10 and SEQ ID NO:11 is amplified to obtain a 222 bp fragment containing 6 AT repetitive elements and a 232 bp fragment containing 12 AT repetitive elements;
and/or the primer group of SEQ ID NO:14 and SEQ ID NO:15 is amplified to obtain a 217 bp fragment containing 5 GAT repetitive elements and a 223 bp fragment containing 7 GAT repetitive elements, the Floccularia luteovirens is judged as Floccularia luteovirens with high total polysaccharide content.

6. The method for screening Floccularia luteovirens with the total polysaccharide content index according to claim 4, wherein a reaction system of the fluorescent PCR amplification reaction in step S2 is:
5 μL of 2×Taq PCR Master Mix, 1 μL of genomic DNA, 0.1 μL of forward primer, 0.4 μL of reverse primer, and 0.4 μL of M13 primer with fluorescence, wherein a volume is fixed to 10 μL with sterile deionized water.

7. The method for screening Floccularia luteovirens with the total polysaccharide content index according to claim 6, wherein the concentrations of the forward primer, the reverse primer and the M13 primer with fluorescence are all 10 uM.

8. The method for screening Floccularia luteovirens with the total polysaccharide content index according to claim 4, wherein a fluorescent PCR amplification reaction procedure in step S2 is:
performing pre-denaturation at 95° C. for 3 min, denaturation at 95° ° C. for 30 s, PCR annealing during the decrease from 62° C. to 55° C. for 30 s, and extension at 72° C. for 30 s, with a total of 10 cycles; performing denaturation at 95° C. for 30 s, annealing at 52° C. for 30 s, and extension at 72° C. for 30 s, with a total of 25 cycles; performing final extension at 72° C. for 20 min; and after heat preservation at 4° C. for 6 h, using the product for fluorescence capillary electrophoresis detection.

9. An application of the DNA barcode according to claim 1 in preparation of a product for screening Floccularia luteovirens with a total polysaccharide content index.

10. An application of the primer group according to claim 2 in preparation of a product for screening Floccularia luteovirens with a total polysaccharide content index.

11. A product for screening Floccularia luteovirens with a total polysaccharide content index, comprising one or more of the primer groups according to claim 2 and satisfying one or more of the following standards:
the primer group of SEQ ID NO:1 and SEQ ID NO:2 is amplified to obtain a 263 bp fragment containing 5 ACC repetitive elements, a 266 bp fragment containing 6 ACC repetitive elements, and a 269 bp fragment containing 7 ACC repetitive elements;
and/or the primer group of SEQ ID NO:6 and SEQ ID NO:7 is amplified to obtain a 255 bp fragment containing 7 TG repetitive elements and a 257 bp fragment containing 8 TG repetitive elements;
and/or the primer group of SEQ ID NO:10 and SEQ ID NO:11 is amplified to obtain a 222 bp fragment containing 6 AT repetitive elements and a 232 bp fragment containing 12 AT repetitive elements;
and/or the primer group of SEQ ID NO:14 and SEQ ID NO:15 is amplified to obtain a 217 bp fragment containing 5 GAT repetitive elements and a 223 bp fragment containing 7 GAT repetitive elements.


SEQUENCE LISTING (17 sequences)

SEQ ID NO: 1 - DNA, 38 bp, synthetic construct
  source 1..38: mol_type = other DNA
tgtaaaacgacggccagtcaccgcaccatctgctgata

SEQ ID NO: 2 - DNA, 20 bp, synthetic construct
  source 1..20: mol_type = other DNA
ggtgattcgtcagtgtgggt

SEQ ID NO: 3 - DNA, 263 bp, synthetic construct
  source 1..263: mol_type = other DNA
caccgcaccatctgctgataatgaatacttgtaccaccaccaccaccaacaccacgcccactcgttttcctgcgtttccctgcggcacattttcctcaatcacaatcacagtcaccgcttccacagtccctcctagtcttgatgttgctcatttccctgtgtggtctcgtttcttggtacacacaggaccgccaccgacctcccgccacctctccgtcatttaacttcccgttatatgctcatacccacactgacgaatcacc

SEQ ID NO: 4 - DNA, 266 bp, synthetic construct
  source 1..266: mol_type = other DNA
caccgcaccatctgctgataatgaatacttgtaccaccaccaccaccaccaacaccacgcccactcgttttcctgcgtttccctgcggcacattttcctcaatcacaatcacagtcaccgcttccacagtccctcctagtcttgatgttgctcatttccctgtgtggtctcgtttcttggtacacacaggaccgccaccgacctcccgccacctctccgtcatttaacttcccgttatatgctcatacccacactgacgaatcacc

SEQ ID NO: 5 - DNA, 269 bp, synthetic construct
  source 1..269: mol_type = other DNA
caccgcaccatctgctgataatgaatacttgtaccaccaccaccaccaccaccaacaccacgcccactcgttttcctgcgtttccctgcggcacattttcctcaatcacaatcacagtcaccgcttccacagtccctcctagtcttgatgttgctcatttccctgtgtggtctcgtttcttggtacacacaggaccgccaccgacctcccgccacctctccgtcatttaacttcccgttatatgctcatacccacactgacgaatcacc

SEQ ID NO: 6 - DNA, 38 bp, synthetic construct
  source 1..38: mol_type = other DNA
tgtaaaacgacggccagttaactatttcccggctcggc

SEQ ID NO: 7 - DNA, 20 bp, synthetic construct
  source 1..20: mol_type = other DNA
gaaaggaaggttcgcgcttg

SEQ ID NO: 8 - DNA, 255 bp, synthetic construct
  source 1..255: mol_type = other DNA
taactatttcccggctcggctctgcagacaaatttcagggtgttccttttcctgatccatacatgccgaaccacacctgtgacctgccgtgcactatgccgataaacttcatcgtttatccaagtctctcccacaggagttatattcgagactccgtgtaaaccttatgtacgtcacaagtatgccaagtagtcagcttcttcgcatgtgtgtgtgtgtgcggtttgtgctaaatcaagcgcgaaccttcctttc

SEQ ID NO: 9 - DNA, 257 bp, synthetic construct
  source 1..257: mol_type = other DNA
taactatttcccggctcggctctgcagacaaatttcagggtgttccttttcctgatccatacatgccgaaccacacctgtgacctgccgtgcactatgccgataaacttcatcgtttatccaagtctctcccacaggagttatattcgagactccgtgtaaaccttatgtacgtcacaagtatgccaagtagtcagcttcttcgcatgtgtgtgtgtgtgtgcggtttgtgctaaatcaagcgcgaaccttcctttc

SEQ ID NO: 10 - DNA, 38 bp, synthetic construct
  source 1..38: mol_type = other DNA
tgtaaaacgacggccagtagcattccagagacgtcagc

SEQ ID NO: 11 - DNA, 20 bp, synthetic construct
  source 1..20: mol_type = other DNA
agacgtcaagatggactgcg

SEQ ID NO: 12 - DNA, 222 bp, synthetic construct
  source 1..222: mol_type = other DNA
agcattccagagacgtcagcgtctgtcatattgataaagttaaagtaatttgtgcttgaagtcatatcatataactattcggcagctcaaagactatgaatgtgcttaccggtaccctagagtatattgaataagattgaattatatatatatacatatataataatggaacgcacacatatccgtattcagagcgatgatacgcagtccatcttgacgtct

SEQ ID NO: 13 - DNA, 232 bp, synthetic construct
  source 1..232: mol_type = other DNA
agcattccagagacgtcagcgtctgtcatattgataaagttaaagtaatttgtgcttgaagtcatatcatataactattcgcagctcaaagactatgaatgtgcttaccggtaccctagagtatattgaataagattgaattatatatatatatatatatatatacatatataataatgaacgcacacatatccgtattcagagcgatgatacgcagtccatcttgacgtct

SEQ ID NO: 14 - DNA, 38 bp, synthetic construct
  source 1..38: mol_type = other DNA
tgtaaaacgacggccagtgaagtggtagtgggactggc

SEQ ID NO: 15 - DNA, 20 bp, synthetic construct
  source 1..20: mol_type = other DNA
tggaagggatagcgaggtga

SEQ ID NO: 16 - DNA, 217 bp, synthetic construct
  source 1..217: mol_type = other DNA
gaagtggtagtgggactggcgaagaagtcagcgaaacccggaggaaggaggaagaagaggaaggaggaggatgtaggagatgatgatgaagaggaagatgatgatgatgatgacgatgatgatgaactttggagaggaagaacatccatctttccggacgcgcttctggctcgatgtccagtctcggccacccaatttcacctcgctatcccttcca

SEQ ID NO: 17 - DNA, 223 bp, synthetic construct
  source 1..223: mol_type = other DNA
gaagtggtagtgggactggcgaagaagtcagcgaaacccggaggaaggaggaagaagaggaaggaggaggatgtaggagatgatgatgaagaggaagatgatgatgatgatgatgatgacgatgatgatgaactttggagaggaagaacatccatctttccggacgcgcttctggctcgatgtccagtctcggccacccaatttcacctcgctatcccttcca